(19) United States
(12) Patent Application Publication
(10) Pub. No.: US 2024/0038326 A1
(43) Pub. Date: Feb. 1, 2024

(54) METHOD AND SYSTEM FOR PHENOTYPIC PROFILE SIMILARITY ANALYSIS USED IN DIAGNOSIS AND RANKING OF DISEASE-DRIVING FACTORS

(71) Applicant: KONINKLIJKE PHILIPS N.V., EINDHOVEN (NL)

(72) Inventors: Yee Him CHEUNG, Boston, MA (US); Jie Wu, Cambridge, MA (US); Nevenka Dimitrova, Pelham Manor, NY (US)

(21) Appl. No.: 17/779,896

(22) PCT Filed: Nov. 20, 2020
(86) PCT No.: PCT/EP2020/082792

§ 371 Date: May 25, 2022

(51) Int. Cl. G16B 20/00; G16B 40/20

(52) U.S. Cl. CPC G16B 20/00; G16B 40/20

(57) ABSTRACT

A method (100) for characterizing a relevance of one or more genes or pathways to a disease of an individual, comprising: (i) obtaining (110) a phenotype profile for the individual, comprising phenotypic characteristics, and differential gene and protein expression information; (ii) identifying (120) one or more database of stored phenotype profiles similar to the individual phenotype profile; (iii) determining (130) a relevance of a genetic pathway to the individual phenotype profile, based at least in part on a similarity between the genetic pathway's known disease/phenotype associations and a phenotype profile of the individual; (iv) determining (140) a relevance of a gene to the individual phenotype profile, based at least in part on a similarity between the gene's known disease/phenotype associations and a phenotype profile of the individual; and (v) reporting (150) one or more genetic pathways and/or one or more genes most relevant to the individual phenotype profile.

### FIELD OF THE DISCLOSURE

The present disclosure is directed generally to methods and systems to characterize the relevance of genes and/or pathways based on phenotype similarity analysis.

### BACKGROUND

As technology for utilizing different types of molecular information becomes more accessible at a lower cost, it is becoming more common to generate multiple types of -omic data (e.g., genomic, transcriptomic, proteomic, and epigenomic) for the same sample. This enables better understand the workings of the underlying complex biological system. The launch of commercial assays such as the NanoString® Vantage 3D and the Illumina® TruSight Tumor 170, based respectively on nCounter® and next-generation sequencing (NGS) technologies, which support the simultaneous extraction of DNA, RNA, and even protein data, pushes further the demand for multi-omic data analysis.

One potential use of multi-omic data analysis is to determine the genetic causes or associations of phenotypes, including disease. Multi-omic data analysis and phenotype comparison would enable analysis at different molecular levels to reveal the mechanism(s) that involve conditions such as genomic aberrations, epigenetic factors, cis/trans-acting gene regulation, and/or gene pathway activation/suppression, which together result in phenotypic or disease manifestation. However, current mechanisms for phenotype analysis and comparison fail to account for sufficiently different potential impacts on a phenotype, and therefore fail to uncover all of the variants and other genomic contributors to disease.

### SUMMARY OF THE DISCLOSURE

There is a continued need for methods and systems that identify more causal variants in a genetic sample. The present disclosure is directed to inventive methods and systems for identifying causal variants in a genetic sample based on the aggregate evidence of multi-level functional impacts established on several types of -omic data. Various embodiments and implementations herein are directed to a system and method that identifies one or more database of stored phenotype profiles similar to the individual phenotype profile. The system determines a relevance of one or more genetic pathways to the individual phenotype profile, based at least in part on a similarity between the genetic pathway's known disease/phenotype associations and a phenotype profile of the individual. The system also determines a relevance of one or more genes to the individual phenotype profile, based at least in part on a similarity between the gene's known disease/phenotype associations and a phenotype profile of the individual.

By applying integrative analysis on multi-omic data of individual patient samples, causal variants in each patient sample are more effectively identified with a higher ranking that is based on the aggregate evidence of multi-level functional impacts established on multi-omic data. Such an approach also assists users to investigate more thoroughly the molecular mechanism of a disease or other phenotype under study.

Generally, in one aspect, is a method for characterizing a relevance of one or more genes or pathways to a disease of an individual using a relevance analysis system. The method includes: (1) obtaining a phenotype profile for the individual, comprising one or more phenotypic characteristics of the target individual, differential gene expression information from the target individual, and differential protein expression information from the target individual; (ii) identifying, using a database of stored phenotype profiles, one or more database of stored phenotype profiles, such as those associated with specific diseases, similar to the individual phenotype profile; (iii) determining a relevance of one or more genetic pathways to the individual phenotype profile, based at least in part on a similarity between the genetic pathway's known disease/phenotype associations and a phenotype profile of the individual; (vi) determining a relevance of one or more genes to the individual phenotype profile, based at least in part on a similarity between the gene's known disease/phenotype associations and a phenotype profile of the individual; and (v) reporting one or more genetic pathways and/or one or more genes most relevant to the individual phenotype profile.

According to an embodiment, the phenotype profile for the individual further comprises a weight for one or more of the phenotypic characteristics of the target individual.

According to an embodiment, identifying one or more database of stored phenotype profiles similar to the individual phenotype profile comprises a similarity score for each pairwise comparison between the individual phenotype profile and the stored phenotype profiles.

According to an embodiment, identifying one or more database of stored phenotype profiles similar to the individual phenotype profile comprises selecting one or more stored phenotype profiles with a highest similarity score.

According to an embodiment, determining a relevance of one or more genetic pathways to the individual phenotype profile comprises identifying one or more genetic pathways potentially associated with one or more phenotypic characteristics of the individual.

According to an embodiment, determining a relevance of one or more genetic pathways to the individual phenotype profile comprises exclusion of any pathway where a detected activity of the pathway and an expected activity of the pathway are opposite directions.

According to an embodiment, determining a relevance of one or more genes to the individual phenotype profile comprises identifying one or more genes potentially associated with one or more phenotypic characteristics of the individual.

According to an embodiment, determining a relevance of one or more genes to the individual phenotype profile comprises exclusion of any gene where a detected activity of the gene and an expected activity of the gene are opposite directions.

According to an aspect is a system configured to characterize a relevance of one or more genes or pathways to a disease of an individual. The system comprises: a phenotype profile for the individual, comprising one or more phenotypic characteristics of the target individual, differential gene expression information from the target individual, and differential protein expression information from the target individual; and a processor configured to: (i) identify, using a database of stored phenotype profiles, one or more database of stored phenotype profiles similar to the individual phenotype profile; (ii) determine a relevance of one or more genetic pathways to the individual phenotype profile, based at least in part on a similarity between the genetic pathway's known disease/phenotype associations and a phenotype profile of the individual; (iii) determine a relevance of one or more genes to the individual phenotype profile, based at least in part on a similarity between the gene's known disease/phenotype associations and a phenotype profile of the individual; and (iv) report one or more genetic pathways and/or one or more genes most relevant to the individual phenotype profile.

According to an embodiment, the system further includes a user interface configured to provide the report of one or more genetic pathways and/or one or more genes most relevant to the individual phenotype profile.

According to an aspect is a method for identifying one or more stored phenotype profiles similar to a query phenotype profile. The method includes: (i) generating or obtaining a weight for a query phenotype profile; (ii) comparing the weighted query phenotype profile to a database of weighted stored phenotype profiles; (iii) identifying at least one weighted stored phenotype profile similar to the weighted query phenotype profile; (iv) performing a weighting function to combine the weights of the weighted query phenotype profile and the at least one weighted stored phenotype profile, comprising creation of a similarity score and a determination of the effective number of matching phenotypic terms between the weighted query phenotype profile and the at least one weighted stored phenotype profile; (v) performing an association test on the similarity score and the number of matching phenotypic terms to determine a similarity value and/or a p-value comprising a statistical significance of the association between the two profiles; and (vii) reporting the at least one weighted stored phenotype profile and its determined similarity value and/or p-value.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for characterizing the relevance of genes and/or pathways based on phenotype similarity analysis, in accordance with an embodiment.

FIG. 2 is a flowchart of a method for identifying one or more phenotype profiles in a database as being similar to the generated phenotype profile, in accordance with an embodiment.

FIG. 3 is a flowchart of a method for determining the relevance of one or more genetic pathways to the phenotype, in accordance with an embodiment.

FIG. 4 is a flowchart of a method for determining the relevance of one or more genes to the phenotype, in accordance with an embodiment.

FIG. 5 is a flowchart of a method for characterizing relevance of genes and/or pathways based on phenotype similarity analysis using a relevance analysis system, in accordance with an embodiment.

FIG. 6 is a schematic representation of a relevance system, in accordance with an embodiment.

### DETAILED DESCRIPTION OF EMBODIMENTS

The present disclosure describes various embodiments of a system and method to characterize the relevance of genes and/or pathways based on phenotype similarity analysis. More generally, Applicant has recognized and appreciated that it would be beneficial to provide a method that characterize a relevance of one or more genes or pathways to a disease of an individual using a relevance analysis system. The system obtains a phenotype profile for the individual, comprising one or more phenotypic characteristics of the target individual, differential gene expression information from the target individual, and differential protein expression information from the target individual. The system identifies one or more database of stored phenotype profiles similar to the individual phenotype profile. The system determines a relevance of one or more genetic pathways to the individual phenotype profile, based at least in part on a similarity between the genetic pathway's known disease/phenotype associations and a phenotype profile of the individual. The system determines a relevance of one or more genes to the individual phenotype profile, based at least in part on a similarity between the gene's known disease/phenotype associations and a phenotype profile of the individual. The system optionally reports one or more genetic pathways and/or one or more genes most relevant to the individual phenotype profile.

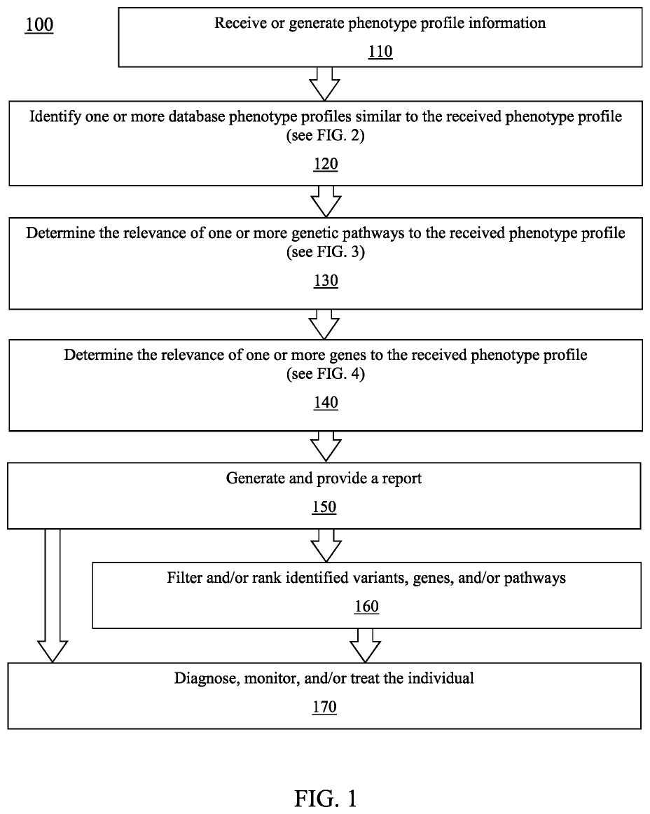

Referring to FIG. 1, in one embodiment, is a flowchart of a method 100 to characterize the relevance of one or more gene and/or pathway based on phenotype similarity analysis using a phenotype analysis system. The phenotype analysis system may be any of the systems described or otherwise envisioned herein, and may comprise any of the components described or otherwise envisioned herein.

At step 110 of the method, a phenotype profile (phen_1) is received. The phenotype profile can be derived from, generated from, or obtained from any source, including a local or remote database of phenotypes and/or phenotypic information. The phenotype profile for the target individual comprises one or more phenotype characteristics of the target individual, differential gene expression information from the target individual, differential protein expression information from the target individual, and/or other information. For example, the target individual may comprise a person of study, such as an individual suffering from a disease that may or may not have a genetic component. Other examples of target individuals include individuals involved in non-disease-related studies where genetic components of a particular phenotype are the object of study. The phenotype characteristics of the target individual can be any phenotypic component, such as a condition of the disease or the particular phenotype.

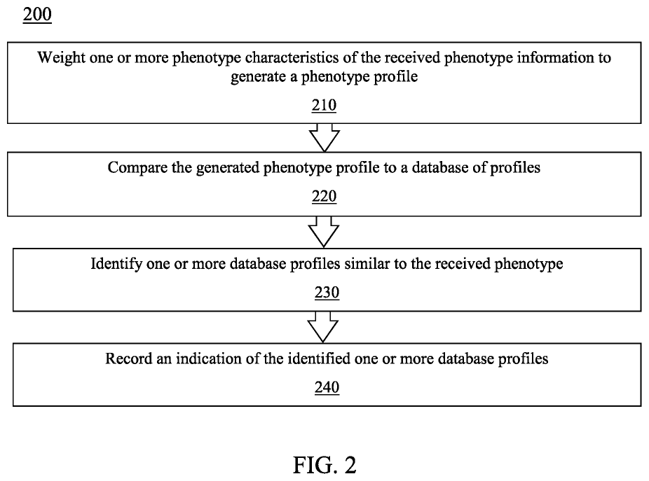

At step 120 of the method, the system identifies one or more phenotype profiles in a database as being similar to the generated phenotype profile. Referring to FIG. 2 is a flowchart of a method (200) for identifying one or more phenotype profiles in a database as being similar to the generated phenotype profile.

At step 210 of the method, one or more of the phenotype characteristics for the received phenotype are weighted. The weighting can comprise any method of weighting known in the art. According to an embodiment, a weight for a phenotype characteristic can be a value between −1 and 1, where the magnitude indicates the degree of manifestation of a phenotype characteristic, and a negative value indicates a negation of a phenotype characteristic. A weight for a phenotype characteristic can be assigned by a user of the system such as a clinician based on their observation and on diagnostic analysis of that phenotype characteristic. Alternatively, and/or additionally, the weight for a phenotype characteristic can be assigned by the system based on diagnostic analysis of that phenotype characteristic. The diagnostic analysis of a phenotype characteristic may comprise data from any observation, testing, or other analysis of the characteristic, including but not limited to imaging data, sensory data, EMR data, and/or other clinical. These weighted phenotype characteristics can be stored in a memory or other data structure, and each will be associated in that data structure with the received phenotype for the target individual.

According to an embodiment, weighting one of more of the phenotype characteristics of the received phenotype for the target individual results in a generated phenotype profile (phen_1, weight_1). This generated phenotype profile, optionally stored in a memory or other data structure, is utilized in further steps of the method.

At step 220 of the method, the system compares the generated phenotype profile to a plurality of phenotype profiles in a database. The goal is to evaluate the resemblance of the generated phenotype profile to one or more of the plurality of phenotype profiles in the database. The database comprises a plurality of phenotype profiles which can be from any source. According to one embodiment, the plurality of phenotype profiles in the database comprises phenotypes for a plurality of different traits, diseases, and other conditions.

According to one embodiment, the database optionally comprises the similarity of all phenotype pairs, with 1 for an exact match and 0 for a complete mismatch between the two phenotypes in the pair. Since in most cases the phenotype pairs are completely unrelated, only those with non-zero similarity scores need to be specified. Similarity can also comprise any number between 1 and 0. This can be generated on demand, in batches, or as new phenotype profile is added to the database.

At step 230, the system identifies, based on the comparison in step 220, one or more phenotype profiles in the database that are most similar to the generated phenotype profile. The identification of a similar phenotype profile can be accomplished by any method for comparison of two phenotype profiles. The comparison may or may not consider the weighting of the generated phenotype profile and/or the database phenotype profiles. For example, the system may generate similarity scores for each pairwise comparison between the generated phenotype profile and the database phenotype profiles, and may select one or more of the database phenotype profiles with the highest similarity score. The one or more database phenotype profiles with the highest similarity score can then be used for downstream steps of the method.

According to one non-limiting embodiment, the one or more phenotype profiles in the database that are most similar to the generated phenotype profile can be identified using the following process, although any element of the process may be modified or removed and other elements may be added. Additionally, very different processes may be utilized to identify one or more phenotype profiles in the database that are most similar to the generated phenotype profile. According to this process the following steps are utilized:
For every pair of phenotype characteristics joined from profiles 1 and 2, phen_1[i] and phen_2[j] (where phen 2, weight 2 are the vectors of phenotype characteristics and corresponding weights from a second phenotype profile similar to the first one), where i and j are the indices to the two vectors, compute a score matrix according to the following equation:

score[i,j]=f(weight_1[i],weight_2[j])*s[i,j] (Eq. 1)

where s[i,j] is the pre-defined similarity score between phen_1[i] and phen_2[j]; and f_{w}( ) is a weighting function that takes weight_1[i] and weight_2[j] as inputs. Depending on the assumptions and objectives, the following are some possible definitions of f_{w}( ): (1) f_{w}=weight_1[i]*weight_2[j]; (2) f_{w}=1−absolute(weight_2[i]−weight_1[j]); and (3) f_{w}=1−absolute(max(weight_2[i]−weight_1[j], 0)). Note that f_{w} could be a negative value, which means the corresponding phenotype manifestation is in opposite directions in the two profiles.
Generate a sum_weight_1 and a sum_weight_2 using the equations:

sum_weight_1=sum(absolute(weight_1))

sum_weight_2=sum(absolute(weight_2)) (Eq. 2)
A similar phenotype profile can then be generated by the following process (Loop_1):
   For any i where the row score[i,] are all zeros, remove row i from score, and the i^{th} element of both phen_1 and weight_1;
   For any j where the column score[,j] are all zeros, remove column j from score, and the j^{th} element of both phen_2 and weight_2;
   Find all index pairs {l, m}∈P where score[l, m]==max(score);
   If there is only one index pair in P, then iₙ=l; jₙ=m;
   Else choose the best pair from P that can maximize a user-defined utility function, e.g.
      utility_max=0;
      For each {l, m}∈P
         Compute the next highest possible score for phen_1[l] using y1=max(score[−l, m]) (note that a negative index −m indicates that the column m is excluded from matrix score while keeping all the other columns);
         Compute the next highest possible score for phen_2[m] using y2=max(score[−l, m])
         (Note that a negative index −l indicates that row l is excluded from matrix score while keeping all the other rows);
         utility=(score[l, m]−y1)+(score[l, m]−y2); and
         if utility>utility_max, then iₙ=l; jₘ=m; utility_max=penalty;
   Register in match_results table an entry using a data entry such as: {phen_1[in], phen_2[in], score[in, jn], weight_1[in], weight_2[jn], s[in, jn]};
   Remove row in from score, and the in th element of both phen_1 and weight_1;
   Remove column jn from score, and the jn th element of both phen_2 and weight_2; and
   Repeat from Loop_1 until phen_1 or phen_2 is empty.
Alternatively, it also possible to match the phenotype items based on the similarity matrix s and then compute the score using the following equation:

score[iₙ,jₙ]=f_{w}(weight_1[iₙ],weight_2[jₙ])*s[iₙ,jₙ] (Eq. 3)
match_val=sum of all score entries in match_results; Since f_{w} could be negative, match_val could also be negative, which means the two profiles have opposite overall phenotype manifestations.
match_fract_1=max(match_val, 0)/sum_weight_1;
match_fract_2=max(match_val, 0)/sum_weight_2;
match_mean_geo=\sqrt{match_fract_1−match_fract_2}

$$match\_mean \_har = ( 1 + {β}^{2} ) \frac{match\_fract \_ 1 \cdot match\_fract \_ 2}{( {β}^{2} \cdot match\_fract \_ 1 ) + match\_fract \_ 2} ,$$

where the default value of β is 1 and the returned value is called the harmonic mean of match_fract_1 and match_fract_2. A user can increase (decrease) the magnitude of 6 to weigh match_fract_1 lower (higher) than match_fract_2.
match_mean_ari=(match_fract_1+match_fract_2)/2
Define the following parameters in a confusion matrix:
   (1) N=n_phen (which is the total number of background phenotype entries being considered in the analysis);
   (2) K=round(sum_weight_2);
   (3) n=round(sum_weight_1); and
   (4) k=round(max(match_val, 0));
   where round(x) is a function that rounds x to the closest integer value.

Based on Fisher's exact test, a p value that measures the statistical evidence for the association of the two phenotype profiles can be generated via the equation:

$$p\_val = {\sum }_{x = k}^{min ( K , n )} \frac{( \begin{matrix}K \\ x\end{matrix} ) ( \begin{matrix}N - K \\ n - x\end{matrix} )}{( \begin{matrix}N \\ n\end{matrix} )} \qquad ( Eq . 4 )$$

Alternatively, p val can also be generated based on any other appropriate methods for association tests.

Thus, at step 230 the system identifies and ranks one or more phenotype profiles in the database that are most similar to the generated phenotype profile based on the computed similarity scores and p values.

At step 240 of the method, the identified one or more phenotype profiles in the database that are most similar to the generated phenotype profile are recorded or otherwise noted or persistently identified. For example, the identified one or more phenotype profiles may be stored in data table or other data format or data structure. As another example, a pointer to the identified one or more phenotype profiles may be generated or stored. As another example, an identification of the identified one or more phenotype profiles may be reported, such as via a printed or displayed report. According to an embodiment, the report comprises one or more of:
One or more identified database phenotype profiles (phen_2) similar to the generated phenotype profile, optionally including a value (match_val) that summarizes the effective number of matched database phenotype profiles;
A p value of the association (p_val) between the generated phenotype profile (phen_1) and each of the one or more identified database phenotype profiles (phen_2). According to an embodiment, since the test is for the direction of phenotypic resemblance can be only, the p value should be one-sided and thus can decrease with the number of matched database phenotype profiles;
A fractional value (match_fract_1) that indicates the effective match with reference to a first phenotypic profile;
A fractional value (match_fract_2) that indicates the effective match with reference to a second phenotypic profile;
A value (match_mean_geo) comprising a geometric mean of match_fract_1 and match_fract_2, a value (match_mean_har) comprising a harmonic mean of match_fract_1 and match_fract_2; and/or a value (match_mean_ari) comprising an arithmetic mean of match_fract_1 and match_fract_2;
A data structure (match_results) comprising a table or other data structure or format summarizing the optimum matches between phenotypes from the first and second phenotypic profiles with one or more of the following fields, among other possible fields:
   phen_1—phenotype item from profile 1 that is matched to phen_2;
   phen_2—phenotype item from profile 2 that is matched to phen_1;
   score—a value that measures the relatedness of phen_1 and phen_2 from the two profiles;
   weight_1—weighting for phen_1 as defined in the input data;
   weight_2—weighting for phen_2 as defined in the input data; and/or
   s—a similarity score between phen_1 and phen_2 as defined in the input data.
      Many other fields are possible.

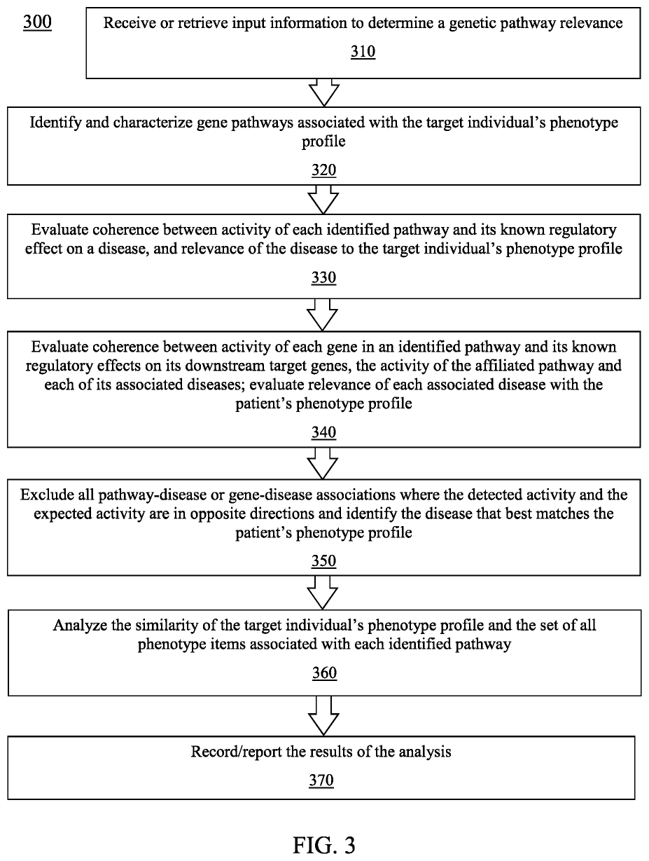

Returning to method 100 in FIG. 1, at step 130 of the method the system determines the relevance of one or more genetic pathways to the phenotype, based on similarity between the genetic pathways' known disease/phenotype associations and the disease/phenotype profile of the target individual. According to an embodiment, the system receives or generates a list of phenotypes of the target individual (patient_phen) by finding a union of the phenotypes that are directly associated with the patient or through the disease-phenotype mappings of their diagnosed diseases. Referring to FIG. 3 is a flowchart of a method (300) for determining the relevance of one or more genetic pathways to the phenotype.

At step 310 of the method, the system receives or retrieves input information to determine genetic pathway relevance to the phenotype of the target individual. The input information comprises, for example, differential gene expression data obtained from a sample from the target individual, differential protein expression data obtained from a sample from the target individual, pathway activity prediction, information about the patient's disease and phenotypes, and information about gene-based expression regulatory status and score for one or more variants obtained from a sample from the target individual. According to an embodiment, the gene-based expression regulatory status and score (gene_reg_results) are modified or otherwise adjusted for copy number variant (CNV) and epigenetic factors as obtained from a sample from the target individual. The gene-based expression regulatory status and score and the copy number variant (CNV) and epigenetic factors, including the process of adjustment, can be obtained via a process described in co-filed U.S. Patent Application No. 62/940,444, the entire contents of which are hereby incorporated herein for all purposes, although other processes are possible.

According to an embodiment, at step 320 of the method, the system identifies one or more gene pathways potentially associated with one or more phenotypes of the target individual, and determines whether the activity of the pathway is neutral, upregulated, or downregulated in the sample from the target individual. The gene pathways potentially associated with one or more phenotypes of the target individual may be identified by the system or otherwise received by the system in step 310. Each gene pathway may comprise a universal or unofficial identification (path id), a name (path name), and a predicted pathway activity score (path activity). According to an embodiment, path_id and path_status can be predefined in external gene pathway databases such as KEGG, Reactome, or Pathway Commons. According to an embodiment, there are existing algorithms used to predict pathway activity scores (path_activity) and the corresponding classifications (path_status) by analyzing the gene expression data of a patient.

According to an embodiment, to determine whether the pathway activity is upregulated, downregulated, or neutral, the system can compare the predicted pathway activity score (path_activity) to a predetermined or user-determined upper boundary or threshold, and a predetermined or user-determined lower boundary or threshold. If the predicted pathway activity score (path activity) is greater than the user-defined upper boundary or threshold, then the pathway activity is identified as being upregulated (path_status=“Up”). If the predicted pathway activity score (path_activity) is lower than the user-defined lower boundary or threshold then the pathway activity is identified as being downregulated (path_status=“Down”). Otherwise, the predicted pathway activity score (path_activity) is identified as being neural (path_status=“Neutral”).

At step 330 of the method, the system performs a phenotypic profile similarity test on a disease identified as being associated with the patient's phenotype based on identified gene pathways. The system first generates a table or other data structure or format (path_disease) comprising a summary of all disease associations of one or more of the identified gene pathway. This can be obtained, for example, from pathway-disease databases such as KEGG, Reactome, and others, with associations between a disease or phenotypes and gene pathways. According to an embodiment, the table (path_disease) comprises one or more of the following pieces of information, although other pieces of information are possible:
An identification (disease_id) and name (disease_name) of an associated disease retrieved from a pathway-disease databases, where values can be “Up”, “Down” or “Unknown”; and
A pathway-disease coherence status (path disease status), which is a categorical variable that indicates if path_status is in agreement with path_disease_dir,
   If the retrieved path_disease_dir=“Unknown” or similar indicator then a path_disease_status value is set as “Unknown Direction”;
   Otherwise if the path_status=“Neutral” or similar indicator then a path_disease_status value is set as “Neutral Pathway Activity”;
   Otherwise if the path_status=path_disease_dir, then a path_disease_status value is set as “Agreed Direction”; and
   Otherwise the path_disease_status is set as “Opposite Direction.”

The system then performs a phenotypic profile similarity test on each disease (disease_id, disease_name) identified as being associated with the patient's phenotype based on the identified gene pathways. The phenotypic profile similarity test can result in a score and pval for the disease, which are then entered into the path_disease table.

At step 340 of the method, the system generates a table or other data structure or format comprising a summary (gene_disease) of the disease associations of all genes in the pathway. This can be obtained, for example, from a gene-disease database such as OMIM among others, with associations between genes and diseases. According to an embodiment, the table or data structure (gene_disease) comprises one or more of the following pieces of information, although other pieces of information are possible:
A gene (gene) affiliated with the pathway retrieved from a pathway database;
The regulatory status (gene_reg_status) of the gene (gene) based on its strongest regulatory influence on its direct downstream targets in the specific pathway as recorded in gene_reg_results, where values can be “Agreed Direction”, “Unknown Direction”, “Non-DE”, “Opposite Direction” and “No Evidence”;
The regulatory status (gene_path_status) of the gene (gene) on the activity of the specific pathway computed based on the differential expression of the gene (gene) and the predicted pathway activity status (path_status):
   If gene is not differentially expressed, then gene_path_status=“Non-DE”; else if path_status=“Neutral”, then gene_path_status=“Neutral Pathway Activity”; else if the regulatory direction of the gene on the pathway is unknown, then gene_path_status=“Unknown Direction”; else if the differential expression of the gene is correctly aligned with (in the same direction as) the pathway activity status, than gene_path_status=“Agreed Direction”; and else gene_path_status=“Opposite Direction.”
disease_id, disease_name=the id and name of a disease associated with gene as retrieved from a gene-disease database;
A regulatory direction of the gene (gene_disease_dir) associated with the disease as retrieved from a gene-disease database;
A gene-disease status (gene_disease_status) for the regulatory effect of the gene on the associated disease (disease_id, disease_name), computed based on the differential expression of the gene and the extracted gene-disease regulatory direction (gene_disease_dir):
   If gene_disease_dir=“Unknown”, then gene_disease_status=“Unknown Direction”; else if gene is not differentially expressed, then gene_disease_status=“Non-DE”; else if (gene is up-regulated and gene_disease_dir=“Up”) or (gene is down-regulated and gene_disease_dir=“Down”), then gene_disease_status=“Agreed Direction”; and else gene_disease_status=“Opposite Direction.”

The system then performs a phenotypic profile similarity test on each disease (disease_id, disease_name) to evaluate its association with the patient's phenotype profile. The phenotypic profile similarity test can result in a score and pval for the disease, which are then entered into the gene_disease table.

At step 350 of the method, all pathway-disease or gene-disease associations where the detected activity and the expected activity are in opposite directions are excluded. For example, based on the information in the table or other data structure or format (path_disease) comprising a summary of all disease associations of one or more of the identified gene pathway and the table or other data structure or format comprising a summary (gene_disease) of the disease associations of all genes in the pathway, all pathway-disease or gene-disease associations with path_disease_status, gene_reg_status, gene_path_status or gene_disease_status being “Opposite Direction” are excluded.

The system then determines the selected disease association with the highest phenotypic profile similarity test score or lowest pval, and the following values associated with the selected disease association are set as follows:
disease=disease associated with the pathway or its affiliated genes that is the best match for the phenotypic profile of the patient;
assoc_disease=the list of genes/pathway associated with disease;
score_disease=the phenotypic profile similarity test score of the disease with regard to the patient's phenotypic profile; and
pval_disease=the phenotypic profile similarity test p value of the disease with regard to the patient's phenotypic profile.

The system thus identifies the set of all phenotype items (phen) associated with the pathway and its affiliated genes, obtained by performing a union merge of all phenotypes associated with the selected diseases based on disease-phenotype databases.

At step 360 of the method, the system performs a phenotypic profile similarity test for the aggregate phenotypes (phen) associated with the specific pathway and the patient's phenotypic profile. The phenotypic profile similarity test can result in a similarity score between the aggregate phenotypes and the overall disease/phenotypic profile of the patient (score_phen), as well as a p-value for the association between the aggregate phenotypes and the overall disease/phenotypic profile of the patient (pval_phen).

At step 370 of the method, the results of the analysis are recorded or otherwise noted or persistently identified. For example, the results may be stored in data table or other data format or data structure. As another example, results may be reported, such as via a printed or displayed report. According to an embodiment, the report comprises one or more of:
path_id, path_name—id and name of the gene pathway;
path_status—predicted pathway activity status, which can be for example “Up”, “Down” or “Neutral”;
path_activity—predicted pathway activity score;
disease—a disease known to be associated with the pathway or its affiliated genes that can match best to the patient's disease/phenotypic profile;
assoc_disease—the list of genes associated with disease; also include the pathway in the list if it has a direct association with disease;
score_disease—a matching score that measures the similarity between disease and the overall disease/phenotypic profile of the patient;
pval_disease—p value for the association between disease and the overall disease/phenotypic profile of the patient;
phen—the set of all phenotype items that are associated with the pathway and its affiliated genes through gene/pathway-disease-phenotype mappings;
score_phen—similarity score between the set of phenotypes for the pathway and the overall disease/phenotypic profile of the patient;
pal_phen—p value for the association between phen and the overall disease/phenotypic profile of the patient;
path_disease—a table that summarizes the pathway-disease associations, which can optionally include the following fields among others:
   disease_id, disease_name—id and name of a disease that is known to be associated directly with the pathway;
   path_disease_dir—the regulatory direction of the pathway that is associated with the disease. Values can be “Up”, “Down” or “Unknown”;
   path_disease_status—a categorical variable that indicates if path_status is in agreement with path_disease_dir. Values can be “Agreed Direction”, “Unknown Direction”, “Neutral Pathway Activity” and “Opposite Direction”;
   score—similarity score between disease and the overall disease/phenotypic profile of the patient; and/or
   pval—p value for the association between disease and the overall disease/phenotypic profile of the patient.
gene_disease—a table that summarizes the disease associations of all genes in the pathway, which can optionally include the following fields among others:
   gene—symbol of a gene that is affiliated with the pathway;
   gene_reg_status—a categorical variable that indicates the strongest type of expression regulatory effect of a gene on its direct gene targets defined for the specific pathway. It can be computed based on gene_reg_results (output of the gene-based expression regulatory status and score module). Values can be “Agreed Direction”, “Unknown Direction”, “Non-DE”, “Opposite Direction” and “No Evidence”;
   gene_path_status—a categorical variable that indicates whether a gene's differential expression is in agreement with the pathway activity status according to the pathway definitions. Values can be “Agreed Direction”, “Unknown Direction”, “Non-DE”, “Neutral Pathway Activity”, and “Opposite Direction”;
   disease_id, disease_name—id and name of a disease associated with gene
   gene_disease_dir—regulatory direction of the gene that is associated with the disease. Values can be “Up”, “Down” or “Unknown”;
   gene_disease_status—a categorical variable that indicates if gene_status is in agreement with gene_disease_dir. Values can be “Agreed Direction”, “Unknown Direction”, “Non-DE” and “Opposite Direction”;
   score—similarity score between disease and the overall disease/phenotypic profile of the patient; and/or
   pval—p value for the association between disease and the overall disease/phenotypic profile of the patient.
      Many other fields are possible.

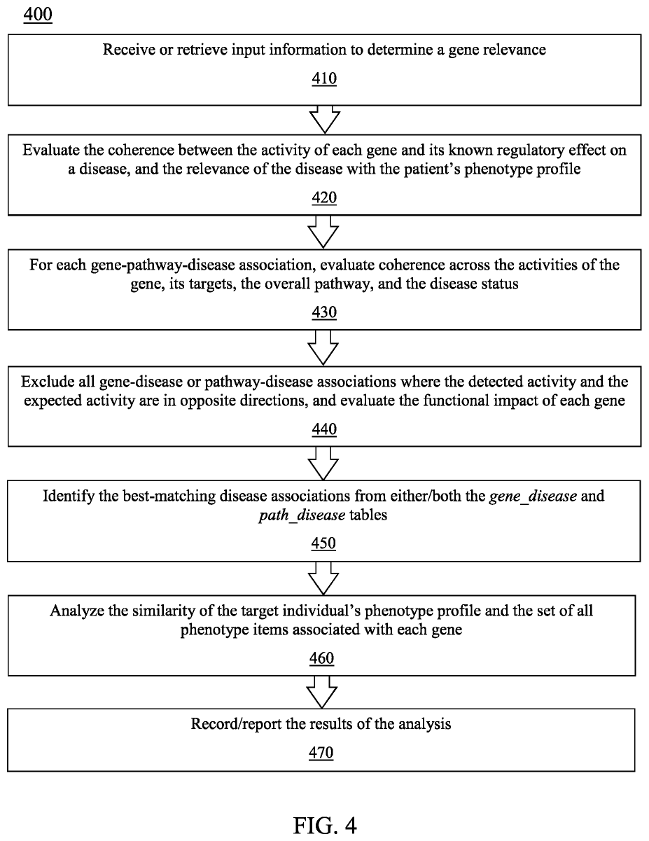

Returning to method 100 in FIG. 1, at step 140 of the method the system determines the relevance of one or more genes to the phenotype profile, based on similarity between the genes' known disease/phenotype associations and the disease/phenotype profile of the target individual. According to an embodiment, the system receives or generates a list of phenotypes of the target individual (patient_phen) by finding a union of the phenotypes that are directly associated with the patient or through the disease-phenotype mappings of their diagnosed diseases. Referring to FIG. 4 is a flowchart of a method (400) for determining the relevance of one or more genes to the phenotype.

At step 410 of method 400, the system receives or retrieves input information to determine gene relevance to the phenotype of the target individual. The input information comprises, for example, differential gene expression data obtained from a sample from the target individual, differential protein expression data obtained from a sample from the target individual, pathway activity prediction, information about the patient's disease and phenotypes, and information about the pathway relevance obtained in step 130 of the method.

According to an embodiment, the system identifies one or more genes potentially associated with one or more phenotypes of the target individual, and determines whether the activity of the gene is neutral, upregulated, or downregulated in the sample from the target individual. The genes potentially associated with one or more phenotypes of the target individual may be identified by the system or otherwise received by the system in step 410.

At step 420 of the method, the system performs a phenotypic profile similarity test on each disease associated with a gene and the patient's phenotypic profile. The system first generates a table or other data structure or format (gene_disease) comprising a summary of all disease associations of the gene. This can be obtained, for example, from gene-disease databases with associations between a disease and genes. According to an embodiment, the table (gene_disease) comprises one or more of the following pieces of information, although other pieces of information are possible:
An identification (disease id) and name (disease name) of an associated disease retrieved from a gene-disease database;
A gene-disease regulatory direction (gene_disease_dir) associated with the retrieved disease, which can also be retrieved from the gene-disease database; and
A gene-disease coherence status (gene_disease_status), which is a categorical variable that indicates if the differential expression of the gene is in agreement with gene_disease_dir.
   if the retrieved gene_disease_dir=“Unknown” or similar indicator then a gene_disease_status value is set as “Unknown Direction”;
   Otherwise if gene is not differentially expressed, then the gene_disease_status value is set as “Non-DE”;
   Otherwise if gene is up-regulated and gene_disease_dir=“Up” or gene is down-regulated and gene_disease_dir=“Down”, then the gene_disease_status values is set as “Agreed Direction”; and
   Otherwise the gene_disease_status value is set as “Opposite Direction”.

The system then performs a phenotypic profile similarity test on the disease (disease_id, disease_name) identified as being associated with the patient's phenotype based on the identified gene. The phenotypic profile similarity test can result in a score and pval for the disease, which are then entered into the gene_disease table.

At step 430 of the method, the system generates a table or other data structure or format comprising a summary (path_disease) of the disease associations of all gene pathways in which the gene (gene) is involved. According to an embodiment, the table or data structure (path_disease) comprises one or more of the following pieces of information, although other pieces of information are possible:
The pathway identification, name, predicted activity status, and score of the pathway (path_id, path_name, path_status, path_activity);
The regulatory status (gene_reg_status) of the gene (gene) based on the strongest influence of the gene on its direct downstream targets in the pathway using the gene_reg_results;
The regulatory status (gene_path_status) of the gene (gene) on the activity of the pathway computed based on the differential expression of the gene (gene) and the predicted pathway activity status (path_status):
   If gene is not differentially expressed, then gene path status=“Non-DE”; else if path_status=“Neutral”, then gene_path_status=“Neutral Pathway Activity”; else if the regulatory direction of the gene on the pathway is unknown, then gene_path_status=“Unknown Direction”; else if the differential expression of the gene is correctly aligned with (in the same direction as) the pathway activity status, than gene_path_status=“Agreed Direction”; and else gene_path_status=“Opposite Direction.”
disease_id, disease_name=the id and name of a disease associated with the pathway;
A regulatory direction of the pathway (path_disease_dir) associated with the disease;
A pathway-disease coherence status (path_disease_status), which is a categorical variable that indicates if path_status is in agreement with path_disease_dir
   If path_disease_dir=“Unknown”, then path_disease_status=“Unknown Direction”; else if path_status=“Neutral” then path_disease_status=“Neutral Pathway Activity”; else if path_status=path_disease_dir, then path_disease_status=“Agreed Direction”; else path_disease_status=“Opposite Direction”.

The system then performs a phenotypic profile similarity test on each disease identified as being associated with the patient's phenotype based on the identified genes. The phenotypic profile similarity test can result in a score and pval for the disease, which are then entered into the path_disease table.

At step 440 of the method, all gene-disease or pathway-disease associations where the detected activity and the expected activity are in opposite directions are excluded. For example, based on the information in the table or other data structure or format (gene_disease) comprising a summary of the disease associations of the gene (gene). and the information in the table or other data structure or format (path_disease) comprising a summary of the disease associations of all gene pathways in which the gene (gene) is involved, all gene-disease or pathway-disease associations with gene_disease_status, gene_reg_status, gene_path_status or path_disease_status being “Opposite Direction” are excluded.

According to an embodiment, the system also counts the following based on the table or other data structure or format (path_disease) comprising a summary of all gene pathways in which the gene (gene) is involved: (1) n_path_dys_cn=number of dysregulated gene pathways in which the gene is functional; (2) n_path_dys=number of dysregulated gene pathways in which the gene is involved; and (3) n_path=number of gene pathways in which the gene is involved.

At step 450 of the method, the system selects from both the gene disease and path_disease tables the disease association with the highest phenotypic profile similarity test score or lowest pval, and the following values associated with the selected disease association are set as follows:
disease overall=disease associated with the gene or its affiliated pathway that is the best match for the phenotypic profile of the patient;
score_overall=the phenotypic profile similarity test score of the disease with regard to the patient's phenotypic profile; and
pval_overall=the phenotypic profile similarity test p value of the disease with regard to the patient's phenotypic profile.

Similarly, the system selects from the gene_disease table the best-matching disease association (disease), and its corresponding similarity score (score_disease) and p value (pval_disease).

Similarly, the system identifies the pathway with the best matching disease association based on the selected disease associations from the path_disease table (the summary of the disease associations of all gene pathways in which the gene (gene) is involved).

According to one embodiment, the system identifies the pathways that are dysregulated (path_status=“Up” or “Down”) and with the gene being functional (gene_reg_status< >{“Non-DE”, “Opposite Direction”, “No Evidence”}). From these pathways, the system identifies the best matching disease association with the highest score or lowest p value. The system assigns the id of that pathway, its associated disease, and its phenotypic profile similarity score and p value to the variables path_dys_fcn, disease_path_dys_fcn, score_path_dys_fcn, pval_path_dys_fcn respectively.

According to one embodiment, the system identifies the pathways that are dysregulated (path_status=“Up” or “Down”). From these pathways, the system finds the best matching disease association with the highest score or lowest p value. The system then assigns the id of that pathway, its associated disease, and its phenotypic profile similarity score and p value to the variables path_dys, disease_path_dys, score_path_dys, pval_path_dys respectively.

According to one embodiment, the system identifies the pathway with the best matching disease association with the highest score or lowest p value, and assigns the id of that pathway, its associated disease, and its phenotypic profile similarity score and p value to the variables path, disease_path, score_path, pval_path respectively.

At step 460 of the method, the system identifies the set of all phenotype items (phen) associated with the pathway and its affiliated genes, obtained by performing a union merge of all phenotypes associated with the selected diseases based on disease-phenotype databases. The system then performs a phenotypic profile similarity test for the aggregate phenotypes (phen) of the gene and the patient's phenotypic profile. The phenotypic profile similarity test can result in a similarity score between the aggregate phenotypes and the overall disease/phenotypic profile of the patient (score_phen), as well as a p-value for the association between the aggregate phenotypes and the overall disease/phenotypic profile of the patient (pval_phen).

At step 470 of the method, the results of the analysis are recorded or otherwise noted or persistently identified. For example, the results may be stored in data table or other data format or data structure. As another example, results may be reported, such as via a printed or displayed report. According to an embodiment, the report comprises one or more of the following for each gene:
gene_reg_status—a categorical variable (output of the gene-based expression regulatory status and score module) that indicates the strongest type of expression regulatory effect of a gene on its direct gene targets. Values can be “Agreed Direction”, “Unknown Direction”, “Non-DE”, “Opposite Direction” and “No Evidence”;
n_path_dys_fcn—number of dysregulated gene pathways in which the gene is functional
n_path_dys—number of dysregulated gene pathways in which the gene is involved;
n_path—number of gene pathways in which the gene is involved;
disease_overall, score_overall, pval_overall—disease associated with the gene or its affiliated pathways with correct regulatory directions that matches best to the patient's disease and phenotypes, and the corresponding phenotypic profile similarity test score and p value for that disease;
disease, score_disease, pval_disease—disease directly associated with the gene with correct regulatory directions that matches best to the patient's disease and phenotypes, and the corresponding phenotypic profile similarity test score and p value for that disease;
phen, score_phen, pval_phen—the set of all phenotype items associated with the gene through its disease associations with correct regulatory directions, and the corresponding phenotypic profile similarity test score and p value for that set of phenotypes;
path_dys_fcn, disease_path_dys_fcn, score_path_dys_fcn, pval_path_dys_fcn—the specific gene pathway that is dysregulated, in which the gene is functional, and associated with a disease that matches best to the patient's disease and phenotypes with correct regulatory directions, the best matching disease associated with that pathway, and its phenotypic profile similarity test score and p value;
path dys, disease path dys, score path dys, pval path dys—the specific gene pathway that is dysregulated (regardless of whether the gene is functional or not) and associated with a disease that matches best to the patient's disease and phenotypes with correct regulatory directions, the best matching disease associated with that pathway, and its phenotypic profile similarity test score and p value;
path, disease_path, score_path, pval_path—the specific gene pathway (dysregulated or not) and associated with a disease that matches best to the patient's disease and phenotypes with correct regulatory directions, the best matching disease associated with that pathway, and its phenotypic profile similarity test score and p value;
gene disease—a table that summarizes all disease associations of the gene with one or more of the following fields:
   disease_id, disease_name=id and name of an associated disease retrieved from the gene-disease database;
   gene_disease_dir—gene regulatory direction associated with the disease, which can be retrieved from the gene-disease database. Values can be “Up”, “Down” or “Unknown”;
   gene_disease_status—a categorical variable that indicates if the differential expression (up/down) of the gene is in agreement with gene_disease_dir. Values can be “Agreed Direction”, “Unknown Direction”, “Non-DE” and “Opposite Direction”; and
   score and pval for the disease by applying phenotypic profile similarity test on the disease and the patient's phenotypic profile or other methods
path_disease—a table that summarizes the disease associations of all pathways in which the gene is involved, with one or more of the following fields:
   path_id, path_name—id and name of a gene pathway;
   path_status—predicted pathway activity status, which can be “Up”, “Down” or “Neutral”;
   path_activity—predicted pathway activity score;
   gene_reg_status—a categorical variable that indicates the strongest type of expression regulatory effect of a gene on its direct gene targets defined for this specific pathway. It can be computed based on gene_reg_results (output of the gene-based expression regulatory status and score module). Values can be “Agreed Direction”, “Unknown Direction”, “Non-DE”, “Opposite Direction” and “No Evidence”;
   gene_path_status—a categorical variable that indicates whether a gene's differential expression is in agreement with the pathway activity status according to the pathway definitions. Values can be “Agreed Direction”, “Unknown Direction”, “Non-DE”, “Neutral Pathway Activity”, and “Opposite Direction”;
   disease_id, disease_name—id and name of a disease associated with this pathway;
   path_disease_dir—regulatory direction of the pathway that is associated with the disease. Values can be “Up”, “Down” or “Unknown”;
   path_disease_status—a categorical variable that indicates if path_status is in agreement with path_disease_dir. Values can be “Agreed Direction”, “Unknown Direction”, “Neutral Pathway Activity” and “Opposite Direction”;
   score—similarity score between disease and the overall disease/phenotypic profile of the patient; and
   pval—p value for the association between disease and the overall disease/phenotypic profile of the patient.

At step 150 of the method, the system generates a report comprising the finalized information. This can comprise storing the information in a data table or other data format, or via a printed or displayed report.

At step 160 of the method, a user may filter and/or rank a plurality of variants, genes, and/or pathways identified by the method, based at least in part on one or more statuses or scores generated as described or otherwise envisioned herein. As one example, the system may create and report a list of variants, genes, and/or pathways that are identified as comprising a particular effect, and rank them according to the likelihood of the potential strength of that impact.

At step 170 of the method, according to an embodiment, a healthcare professional, researcher, or other user may receive the report generated by the system and comprising any of the information described or otherwise envisioned herein, and utilize that report to diagnose, monitor, and/or treat the individual. For example, the receiving individual can review the report and identify one or more variants, genes, and/or pathways identified in the report as being likely to be involved in the test-taker's phenotype, and therefore likely targets for treatment and/or intervention. According to one embodiment, once an identification is made the receiving individual or a person acting on behalf of the receiving individual implements a treatment or intervention to treat the phenotype. This may include a specific medical treatment based on a known association between the identified variants, genes, and/or pathways and specific medicines or interventions, for example. According to another embodiment, once an identification is made the receiving individual or a person acting on behalf of the receiving individual can utilize the information for research purposes to identify potential treatment and/or interventions. Thus there can be a direct relationship between the variants, genes, and/or pathways, the output of the analytical method and system that examines the variants, genes, and/or pathways, and the treatment or study of the individual.

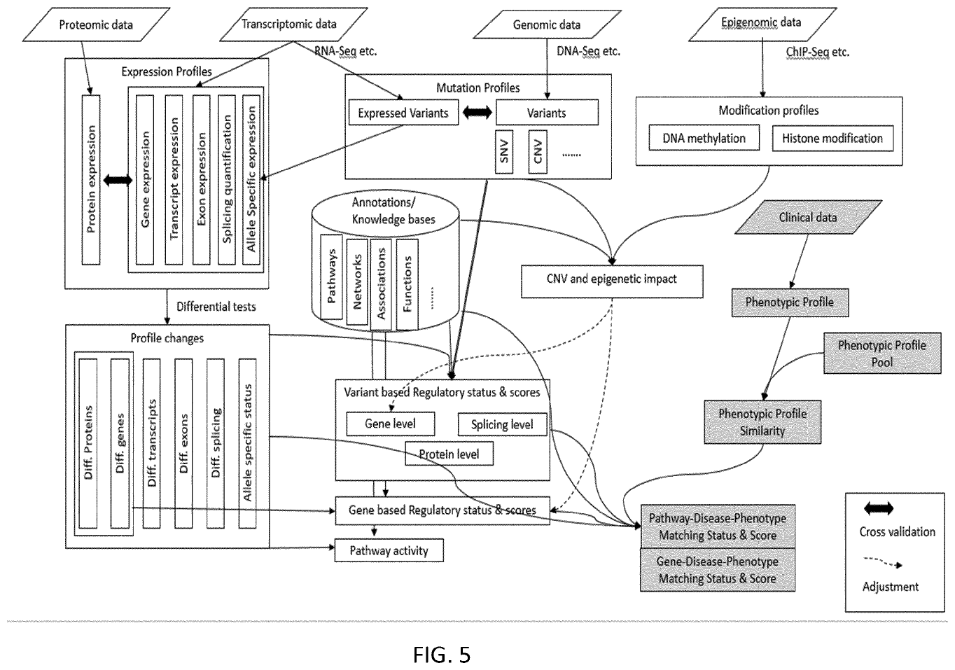

Referring to FIG. 5, in one embodiment, is a flowchart of a method 700 for characterizing relevance of genes and/or pathways based on phenotype similarity analysis using a relevance analysis system. The relevance analysis system may be any of the systems described or otherwise envisioned herein, and may comprise any of the components described or otherwise envisioned.

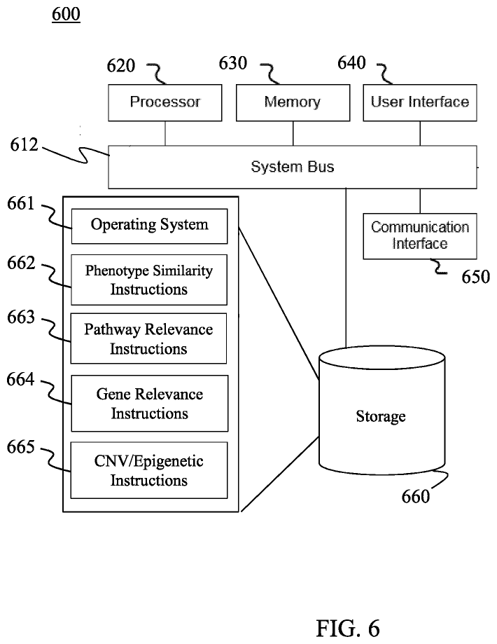

Referring to FIG. 6, in one embodiment, is a schematic representation of a relevance analysis system 600 configured to characterize the functional impact of genomic variants identified from a genomic sample. System 600 may be any of the systems described or otherwise envisioned herein, and may comprise any of the components described or otherwise envisioned herein.

According to an embodiment, system 600 comprises one or more of a processor 620, memory 630, user interface 640, communications interface 650, and storage 660, interconnected via one or more system buses 612. It will be understood that FIG. 6 constitutes, in some respects, an abstraction and that the actual organization of the components of the system 600 may be different and more complex than illustrated.

According to an embodiment, system 600 comprises a processor 620 capable of executing instructions stored in memory 630 or storage 660 or otherwise processing data to, for example, perform one or more steps of the method. Processor 620 may be formed of one or multiple modules. Processor 620 may take any suitable form, including but not limited to a microprocessor, microcontroller, multiple microcontrollers, circuitry, field programmable gate array (FPGA), application-specific integrated circuit (ASIC), a single processor, or plural processors.

Memory 630 can take any suitable form, including a non-volatile memory and/or RAM. The memory 630 may include various memories such as, for example L1, L2, or L3 cache or system memory. As such, the memory 630 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 600. It will be apparent that, in embodiments where the processor implements one or more of the functions described herein in hardware, the software described as corresponding to such functionality in other embodiments may be omitted.

User interface 640 may include one or more devices for enabling communication with a user. The user interface can be any device or system that allows information to be conveyed and/or received, and may include a display, a mouse, and/or a keyboard for receiving user commands. In some embodiments, user interface 640 may include a command line interface or graphical user interface that may be presented to a remote terminal via communication interface 650. The user interface may be located with one or more other components of the system, or may located remote from the system and in communication via a wired and/or wireless communications network.

Communication interface 650 may include one or more devices for enabling communication with other hardware devices. For example, communication interface 850 may include a network interface card (NIC) configured to communicate according to the Ethernet protocol. Additionally, communication interface 650 may implement a TCP/IP stack for communication according to the TCP/IP protocols. Various alternative or additional hardware or configurations for communication interface 650 will be apparent.

Storage 660 may include one or more machine-readable storage media such as read-only memory (ROM), random-access memory (RAM), magnetic disk storage media, optical storage media, flash-memory devices, or similar storage media. In various embodiments, storage 660 may store instructions for execution by processor 620 or data upon which processor 620 may operate. For example, storage 660 may store an operating system 661 for controlling various operations of system 600.

It will be apparent that various information described as stored in storage 660 may be additionally or alternatively stored in memory 630. In this respect, memory 630 may also be considered to constitute a storage device and storage 660 may be considered a memory. Various other arrangements will be apparent. Further, memory 630 and storage 660 may both be considered to be non-transitory machine-readable media. As used herein, the term non-transitory will be understood to exclude transitory signals but to include all forms of storage, including both volatile and non-volatile memories.

While relevance system 600 is shown as including one of each described component, the various components may be duplicated in various embodiments. For example, processor 620 may include multiple microprocessors that are configured to independently execute the methods described herein or are configured to perform steps or subroutines of the methods described herein such that the multiple processors cooperate to achieve the functionality described herein. Further, where one or more components of system 600 is implemented in a cloud computing system, the various hardware components may belong to separate physical systems. For example, processor 620 may include a first processor in a first server and a second processor in a second server. Many other variations and configurations are possible.

According to an embodiment, storage 660 of relevance system 600 may store one or more algorithms and/or instructions to carry out one or more functions or steps of the methods described or otherwise envisioned herein. For example, processor 620 may comprise phenotype similarity instructions 662, pathway relevance instructions 663, gene relevance instructions 664, and/or report generation instructions or software 665, among many other algorithms and/or instructions to carry out one or more functions or steps of the methods described or otherwise envisioned herein.

According to an embodiment, phenotype similarity instructions 662 direct the system to identify one or more phenotype profiles in a database as being similar to the generated phenotype profile. Referring to FIG. 2 is a flowchart of a method (200) for identifying one or more phenotype profiles in a database as being similar to the generated phenotype profile.

According to an embodiment, pathway relevance instructions 663 direct the system to determine the relevance of one or more genetic pathways to the phenotype, based on similarity between the genetic pathways' known disease/phenotype associations and the disease/phenotype profile of the target individual. According to an embodiment, the system receives or generates a list of phenotypes of the target individual (patient_phen) by finding a union of the phenotypes that are directly associated with the patient or through the disease-phenotype mappings of their diagnosed diseases. Referring to FIG. 3 is a flowchart of a method (300) for determining the relevance of one or more genetic pathways to the phenotype.

According to an embodiment, gene relevance instructions 664 direct the system to determine the relevance of one or more genes to the phenotype, based on similarity between the genes' known disease/phenotype associations and the disease/phenotype profile of the target individual. According to an embodiment, the system receives or generates a list of phenotypes of the target individual (patient_phen) by finding a union of the phenotypes that are directly associated with the patient or through the disease-phenotype mappings of their diagnosed diseases. Referring to FIG. 4 is a flowchart of a method (400) for determining the relevance of one or more genes to the phenotype.

According to an embodiment, report generation instructions 664 direct the system to generate a report comprising information about the analysis performed by the system. The report may be generated for any format or output method, such as a file format, a visual display, or any other format. A report may comprise a text-based file or other format comprising the reported information.

The report generation instructions or software 664 may direct the system to store the generated report or information in temporary and/or long-term memory or other storage. This may be local storage within system 600 or associated with system 600, or may be remote storage which received the report or information from or via system 600. Additionally and/or alternatively, the report or information may be communicated or otherwise transmitted to another system, recipient, process, device, and/or other local or remote location.

The report generation instructions or software 664 may direct the system to provide the generated report to a user or other system. For example, the system may visually display information on the user interface, which may be a screen or other display.

One major challenge in genomic research and precision medicine is to identify the mutations and/or genes that actually cause disease symptoms, out of the hundreds and thousands of candidate variants, which is necessary for scientific discovery or identification of potential treatment targets. While standard variant-filtering approaches based on call quality, population allele frequency, gene-model annotation, known disease association, and predicted pathogenicity can narrow down the pool of candidate variants, multi-omic data analysis of gene expression, CNV, epigenetic, and other data is critical for explaining further the molecular mechanism(s) of disease, which sheds light on disease etiology and treatment options.

One use case of the multi-omic data analysis framework described or otherwise envisioned herein is to facilitate the discovery of variants, genes, and/or pathways that cause or influence disease by performing analysis on the DNA and RNA whole exome sequencing (WES) data of hundreds of samples in a genomic study. By comparing the exon/gene/transcript expression between the carrier and non-carriers of each candidate variant, and using external databases (e.g. expression/splicing quantitative trait loci, promoter/enhancer map, etc.), the framework can evaluate whether a variant has any impact on allele-specific expression, alternative splicing, regulation of target genes, gene pathways, and more. The generated variant-based statuses and scores, as described herein, can then be used to filter and rank variants, genes, and/or pathways by their potential functional impacts.

In addition to variant-based functional impact evaluation, scientists may also gain insights on the functional impact of individual genes and/or pathways. This can be done using the framework described or otherwise envisioned herein to analyze the differential gene expressions between the case and control samples. With reference to pathway definitions in external databases such as KEGG, Reactome and Pathway Commons, the framework can evaluate whether a gene has any impact on its immediate/nearby downstream target genes or overall pathway activities. If CNV, methylation, or other epigenetic data are available, the framework can evaluate the combined CNV and epigenetic impact on each gene. This, in combination with the gene expression results, can further indicate if the differential expression of a gene or any regulatory effect is indeed driven by CNV or epigenetic factors. By carefully and systematically considering the multi-layer evidence obtained from the different -omic data, scientists can pinpoint the causal mutations with explanations for their potential influence on gene targets and pathways.

In a similar fashion, clinicians can use the framework described or otherwise envisioned herein to analyze the DNA and RNA WES data to identify the causal disease mutations or genes in a patient. When evaluating variant-based functional impact, if the data of one patient is insufficient, the gene expression data of carriers and non-carriers from other studies can be employed. Using the framework described or otherwise envisioned herein, clinicians can pinpoint the causal mutations and genes with explanations for the molecular mechanism. For example, if a disease is found to be caused by a gene mutation that leads to the up-regulation of the activity of a pathway, then a drug known to suppress the activity of the pathway can be administered to the patient in an attempt to cure the disease or alleviate the symptoms.

Thus, according to an embodiment, the methods and systems described or otherwise envisioned herein comprise many different practical applications. For example, the output of the system or method may be a report comprising one or more of the characterized plurality of statuses and/or scores, among other reports, statuses, and information. This report has many uses, including being used by a physician or other healthcare professional, or a researcher, to determine variants, genes, and/or pathways involved in the phenotype of a particular individual such as a cancer patient or sufferer or a rare genetic disease, among many other possible individuals. The system may generate a report that not only includes a list of variants, genes, and/or pathways likely to be involved in the phenotype of a particular individual, but the report may also comprise a ranking of the most likely variants, genes, and/or pathways, and/or a ranking of the largest impact of likely variants, genes, and/or pathways, and/or a ranking of variants, genes, and/or pathways with the most supporting evidence for impact.

According to another embodiment, the system may be utilized to diagnose conditions. For example, a clinician may observe certain phenotypes and symptoms, but may not be able to make an exact diagnosis based on those observations. Pursuant to the methods and systems described or otherwise envisioned herein, a phenotype profile is created and weights can be applied or generated. The phenotypic profile similarity test described herein can then be utilized to compare the list of phenotypes with a database of phenotype profiles, which are associated with a disease diagnosis or diagnoses. The stored phenotype profile with the highest score or lowest p-value showing the best association with the queried phenotype profile can facilitate a diagnosis and/or additional inquiry. According to an embodiment, one or more of the methods or steps described may be automated. For example, the system may be designed to take images, scans, and/or any other data (temperature, blood pressure, etc.), either directly or from a patient's medical records, and can then determine or generate a list of phenotypes with a level of manifestation, create a phenotype profile with corresponding weights, perform the similarity test, and propose or generate diagnosis or diagnoses, or additional testing. Many other options are possible.

The methods and systems described herein comprise several limitations each comprising and analyzing millions of pieces of information. For example, the variant information and associated expression (and potentially other) information received or generated by the system likely comprises many 1000s of potential variants, genes, pathways, and other points of data for analysis. Similarly, each step of the process comprises analysis of those 1000s of potential variants, genes, pathways, and other points of data, thereby constituting millions of calculations. This is something the human mind is not equipped to perform, even with pen and pencil.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for characterizing a relevance of one or more genes or pathways to a disease of an individual using a relevance analysis system, comprising:
obtaining a phenotype profile for the individual, comprising one or more phenotypic characteristics of the target individual, differential gene expression information from the target individual, and differential protein expression information from the target individual;
identifying, using a database of stored phenotype profiles, one or more database of stored phenotype profiles similar to the individual phenotype profile;
determining a relevance of one or more genetic pathways to the individual phenotype profile, based at least in part on a similarity between the genetic pathway's known disease/phenotype associations and a phenotype profile of the individual;
determining a relevance of one or more genes to the individual phenotype profile, based at least in part on a similarity between the gene's known disease/phenotype associations and a phenotype profile of the individual; and
reporting one or more genetic pathways and/or one or more genes most relevant to the individual phenotype profile.

2. The method of claim 1, wherein the phenotype profile for the individual further comprises a weight for one or more of the phenotypic characteristics of the target individual.

3. The method of claim 1, wherein identifying one or more database of stored phenotype profiles similar to the individual phenotype profile comprises a similarity score for each pairwise comparison between the individual phenotype profile and the stored phenotype profiles.

4. The method of claim 3, wherein identifying one or more database of stored phenotype profiles similar to the individual phenotype profile comprises selecting one or more stored phenotype profiles with a highest similarity score.

5. The method of claim 1, wherein determining a relevance of one or more genetic pathways to the individual phenotype profile comprises identifying one or more genetic pathways potentially associated with one or more phenotypic characteristics of the individual.

6. The method of claim 1, wherein determining a relevance of one or more genetic pathways to the individual phenotype profile comprises exclusion of any pathway where a detected activity of the pathway and an expected activity of the pathway are opposite directions.

7. The method of claim 1, wherein determining a relevance of one or more genes to the individual phenotype profile comprises identifying one or more genes potentially associated with one or more phenotypic characteristics of the individual.

8. The method of claim 1, wherein determining a relevance of one or more genes to the individual phenotype profile comprises exclusion of any gene where a detected activity of the gene and an expected activity of the gene are opposite directions.

9. A system configured to characterize a relevance of one or more genes or pathways to a disease of an individual, comprising:
a phenotype profile for the individual, comprising one or more phenotypic characteristics of the target individual, differential gene expression information from the target individual, and differential protein expression information from the target individual; and
a processor configured to: (i) identify, using a database of stored phenotype profiles, one or more database of stored phenotype profiles similar to the individual phenotype profile; (ii) determine a relevance of one or more genetic pathways to the individual phenotype profile, based at least in part on a similarity between the genetic pathway's known disease/phenotype associations and a phenotype profile of the individual; (iii) determine a relevance of one or more genes to the individual phenotype profile, based at least in part on a similarity between the gene's known disease/phenotype associations and a phenotype profile of the individual; and (iv) report one or more genetic pathways and/or one or more genes most relevant to the individual phenotype profile.

10. The system of claim 9, further comprising a user interface configured to provide the report of one or more genetic pathways and/or one or more genes most relevant to the individual phenotype profile.

11. The system of claim 9, wherein identifying one or more database of stored phenotype profiles similar to the individual phenotype profile comprises a similarity score for each pairwise comparison between the individual phenotype profile and the stored phenotype profiles.

12. The system of claim 9, wherein determining a relevance of one or more genetic pathways to the individual phenotype profile comprises identifying one or more genetic pathways potentially associated with one or more phenotypic characteristics of the individual.

13. The system of claim 9, wherein determining a relevance of one or more genetic pathways to the individual phenotype profile comprises exclusion of any pathway where a detected activity of the pathway and an expected activity of the pathway are opposite directions.

14. The system of claim 9, wherein determining a relevance of one or more genes to the individual phenotype profile comprises identifying one or more genes potentially associated with one or more phenotypic characteristics of the individual.

15. A method for identifying one or more stored phenotype profiles similar to a query phenotype profile, comprising:
generating or obtaining a weight for a query phenotype profile;
comparing the weighted query phenotype profile to a database of weighted stored phenotype profiles;
identifying at least one weighted stored phenotype profile similar to the weighted query phenotype profile;
performing a weighting function to combine the weights of the weighted query phenotype profile and the at least one weighted stored phenotype profile, comprising creation of a similarity score and a determination of the effective number of matching phenotypic terms between the weighted query phenotype profile and the at least one weighted stored phenotype profile;
performing an association test on the similarity score and the effective number of matching phenotypic terms to determine a similarity value and/or a p-value comprising a statistical significance of the association between the two profiles; and
reporting the at least one weighted stored phenotype profile and its determined similarity value and/or p-value.
